(19) United States
(12) Patent Application Publication
(10) Pub. No.: US 2001/0053228 A1
(43) Pub. Date: Dec. 20, 2001

(54) Noise cancellation system for active headsets

(76) Inventor: Owen Jones, Ipswich (GB)

Correspondence Address: Larson & Associates, P.C., 221 East Church Street, Frederick, MD, 21701-5405, US

(21) Appl. No.: 09/911,126

(22) Filed: Jul. 23, 2001

Related U.S. Application Data
(63) Continuation-in-part of application No. 08/912,509, filed on Aug. 18, 1997.

(51) Int. Cl. A61F 11/06; G10K 11/16; H03B 29/00

(52) U.S. Cl. 381/71.6; 381/72; 381/74; 381/77

(57) ABSTRACT

An active headset system is provided with compatibility with existing socket configurations in a way that minimizes the number of electrical connections. The number of connections between the active headset and the remote noise cancellation circuitry is reduced through the use of a common contact, having a controlled impedance, that serves as an input connection to corresponding terminals of the two earphones of the active headset. According to another aspect of the present invention, the transients associated with plugging in or unplugging stereo jack plugs or up to a seven pin connector into an active headset may be overcome by a transient detector in the noise cancellation circuitry. Yet another aspect of the present invention concerns the powering of the noise cancellation circuitry whether the noise cancellation circuitry is placed inside the active headset or at least partially internal to a remote external device.

### BACKGROUND OF THE INVENTION

1. Field of the Invention

This invention relates generally to a noise cancellation system for active headsets, and more particularly to an active headset capable of compatibility with existing socket configurations of an external device and capable of powering active noise cancellation circuitry whether or not resident in the active headset.

2. Background of the Invention

In relatively noisy conditions such as the interior of an aircraft or other areas where external noise interferes with the ability to hear a signal through a regular headset, it is proposed practice to provide gain-adjustable active headsets wherein, at least to some extent, the external noise is detected by a microphone, a noise cancellation signal is generated and cancellation noise is propagated by an earphone to prevent at least some of the external noise, especially lower frequency noise, from reaching the ear. Such active headsets would substantially improve listening conditions for the user compared to the currently provided passive headsets.

Normally, in the implementation of a stereo active headset, the circuitry necessary for generation of the noise cancellation signal is either incorporated in the headset, together with the microphone and earphone in each earpiece, or in a separate box. All of the circuitry necessary for generation of the noise cancellation signal is thus independent from the external device that is responsible for generating an audio signal to the headset. The only connection required is then for the audio signal and this is accomplished from either the headset or separate box with a standard audio lead terminated with a 3.5 mm stereo jack plug that plugs into the stereo socket of the external device. The same type of socket is also used on computer sound cards. Such external devices include the arm rest of an aircraft seat or consumer stereo equipment such as a Walkman®. Headsets that incorporate the noise cancellation circuitry in the headset, together with the microphone and earphone, and the use of a separate box are relatively expensive.

Unfortunately, it is anticipated that, in an aircraft or in consumer stereo equipment such as a Walkman ®, rough handling by users might result in expensive active headsets or in the need for separate boxes that might be easily damaged or broken and, in some instances, stolen. In either case, costly replacement would be necessary. Additionally, with active headsets, batteries must be used to power the active circuitry, and this involves extra bulk and weight, a problem of particular importance if everything is to be built into the headset. Although the power drain of a well designed headset can be kept low enough to allow for an acceptable lifetime from very small batteries, their cost and limited availability renders this an unpopular solution. It would therefore be most advantageous if the means for powering the active headset could be removed from the headset and located remotely therefrom.

It would additionally be advantageous if some or all of the noise cancellation circuitry could be removed from the headset and located remotely. In an aircraft, the noise cancellation circuitry could be remotely located in the arm rest of the passenger seat. For consumer stereo equipment, the noise cancellation circuitry could be remotely located within the consumer stereo equipment such as a Walkman® or on a computer sound card.

In addition to the cost and power concerns of separating the noise cancellation circuitry from the headset, there are compatibility concerns as well. Conventionally in an aircraft utilizing a passive headset, the wanted sound to be heard on the headset is transmitted via a stereo jack plug socket in the arm rest, and the headset is provided with a stereo jack plug which fits into this socket. Disadvantageously, conventional noise cancellation circuitry, if remotely located in the arm rest, would inherently require replacement of the stereo jack socket by an eight pin socket, due to the number of electrical connections which have to be made to the microphones and loudspeakers in the two earpieces of the headset. One proposal has already been put forward to provide such eight pin sockets in the arm rests, which would necessitate the provision of an expensive eight pin connector on an active headset.

In addition to aircraft applications, consumer stereo equipment that utilizes an active headset also has a stereo jack plug. The headset is provided with a stereo jack plug which fits into this socket. Disadvantageously, conventional noise cancellation circuitry, if remotely located within the stereo equipment, would also inherently require replacement of the stereo jack socket by an eight pin socket, due to the number of electrical connections which have to be made to the microphones and loudspeakers in the two earpieces of the headset.

Referring to FIG. 1, a conventional bridge amplifier for the earphone in an active headset is shown. Two such bridge amplifiers are required, one for each headphone, each with two terminals T₁ and T₂ connecting to noise cancellation circuitry. I/P indicates the noise cancellation signal input to each bridge amplifier and ½V_{cc}. indicates one half the rail voltage (power supply voltage). Thus, if the noise cancellation circuitry is remotely located, plug-in connectors are required to provide four connections. Further, FIG. 3 shows a conventional headset arrangement having two microphones, one in each earpiece L and R, and each with gain control provided by the potentiometers (POT). Four terminal connections T₁, T₂, T₃, and T₄ are required. It would be advantageous to reduce the number of required connections between the headset and electronics. This applies for both stereo headsets, just described, as well as for mono headsets which may additionally employ the use of a boom microphone.

There is thus an unmet need in the art to provide noise cancellation circuitry remote from the active headset that does not require replacement of the stereo jack socket by an eight pin socket and whereby the number of connections required can be reduced generally, for either mono or stereo headsets. There is also an unmet need in the art to provide a means of powering the active headset that is removed from the headset and located remotely therefrom.

### SUMMARY OF THE INVENTION

It is an object of the invention to provide noise cancellation system having noise cancellation circuitry located remote from the active headset.

In another object of the invention that the active headset is provided with two stereo jack plugs or a six pin connector for connection to the noise cancellation circuitry.

It is another aspect of the invention that a mono active headset is provided with two mono jack plugs, one stereo jack plug or a 3 or 4 pin connector for connection to the noise cancellation circuitry.

It is another aspect of the invention that a mono headset with boom microphone is provided with a mono and stereo jack plug or a 4 or 5 pin connector for connection to the noise cancellation circuitry.

It is yet another object of the invention to provide a transient detector in the noise cancellation circuitry to overcome noise in the ear due to the generation of transients that occur when plugging in or unplugging the connector of the active headset.

It is further another object of the invention to reduce the number of necessary electrical connections to the active headset by multiplexing the use of connectors that are already present, thereby maintaining plug compatibility with normal headsets.

It is yet another object of the invention to provide a means of powering the active headset that is removed from the active headset and located remotely therefrom.

Therefore, in accordance with the invention, the active headset is provided with appropriate connectivity to provide compatibility with existing socket configurations. In the case of a stereo headset, either two stereo jack plugs or a six pin connector are provided. For a mono headset, two mono jack plugs, one stereo jack plug or a three or four pin connector are provided and in the case of a mono headset with boom mic, one mono and one stereo or a 4 or 5 pin connector may be used. The number of connections between the active headset and the remote noise cancellation circuitry is reduced from eight to six, five or four through the use of a common contact, having a controlled impedance, that serves as an input connection to corresponding terminals of the two earphones of the active headset. The appropriate arrangement of single-ended operational amplifiers prevents roll-off at lower frequencies. Alternately, bootstrapped emitter-follower transistors or operational amplifiers in the noise cancellation circuitry behave as current sources at audio frequencies as well as provide correct bias voltage to the microphones so that the common contact can serve as an input connection to corresponding terminals of the two microphones in the earpiece.

According to another aspect of the present invention, the transients associated with plugging in or unplugging stereo jack plugs or a six pin connector into an active headset may be overcome by a transient detector in the noise cancellation circuitry. The transient detector may comprise a window comparator and a mute logic circuit for muting the output to the earphones if it exceeds a predetermined amplitude level. Additionally, a decoupling capacitor in the active headset will overcome the plugging in/unplugging transient noise problem and at the same time simplify the circuitry because the bootstrapping and muting circuitry would not be required.

Yet another aspect of the present invention concerns the powering of the noise cancellation circuitry whether the noise cancellation circuitry is placed inside the active headset or inside a remote external device. Where the noise cancellation circuitry is placed inside the active headset, the power necessary to power the headset and the noise cancellation circuitry within it can be obtained from the external device that generates an audio signal to the headset. Several approaches for providing power from the external device that generates the audio signal to the headset include the following: increasing the number of contacts on the connector; using a connector that employs a single supply rail; using retractable contacts on the plug; using existing sockets on the external device; using phantom powering techniques to power the active headset; using a pulse width modulation (PWM) amplifier; and using the output audio signal power produced by the external device to power the active headset.

Where, on the other hand, the noise cancellation circuitry is placed inside an external device outside the headset, electrical connections must be made from the external device to the headset. The number of connections on the socket could be increased, but it is preferable to place some of the noise cancellation circuitry inside the headset to reduce the required number of connections so that socket compatibility may be maintained. First, the jack plug or socket on the external device may be used to make electrical contact. Second, the drive signal to the headset earphone may be converted into a PWM drive signal to power the microphone circuitry. Third, a conventional linear audio signal may be superimposed upon a positive DC voltage level that powers the microphone after the audio signal has been filtered. Fourth, a bridge circuit is used to separate the microphone signal from the earphone drive signal and a DC offset is added to the earphone drive signal to power the microphone circuitry. Inside the headset, the DC offset is blocked from the earphone drive signal. This technique requires that the earphone impedance be known in order to separate the microphone signal from the earphone drive signal. The present invention describes how headset identification could be used to identify the particular active headset model, and therefore its impedance value, in addition to its presence. Fifth, a radio frequency carrier signal may be used in a technique to combine the earphone and microphone connections.

### BRIEF DESCRIPTION OF THE DRAWINGS

The novel features believed characteristic of the invention are set forth in the claims. The invention itself, however, as well as the preferred mode of use, and further objects and advantages thereof, will best be understood by reference to the following detailed description of an illustrative embodiment when read in conjunction with the accompanying drawings, wherein:

FIG. 1 shows a bridge amplifier circuit, in accordance with the prior art for driving an earphone in an active headset;

FIGS. 2a, 2b and 2c are amplifier circuit diagrams that illustrate how the number of connections required for earphones may be reduced from four to three, in accordance with the present invention;

FIG. 3 is a circuit diagram, in accordance with the prior art for headset microphones provided with gain control;

FIG. 4a is a bootstrapping circuit diagram that provides a common contact from amplifiers in the noise cancellation circuitry to corresponding terminals of two earphones so that only three terminal connections are necessary between the microphones and the remote noise cancellation circuitry, in accordance with the present invention, FIG. 4b is a mono version of the circuit.

FIGS. 5a and 5b show circuit diagrams that free the common contact of the earphone(s) of the requirement to be at ground potential, in accordance with the present invention;

FIG. 6 illustrates a transient detector circuit, according to a first embodiment for suppressing noise transients in a headset of the present invention;

FIG. 7 illustrates a transient elimination circuit, according to a second embodiment for suppressing noise transients in a headset of the present invention;

FIG. 8a and 8b show a connector using a single supply rail to supply power to the active headset, in accordance with a first embodiment of the present invention in which the noise cancellation circuitry is placed inside the active headset;

FIG. 9 shows a connector using switch contacts to supply power to the active heads, in accordance with an second embodiment of the present invention in which the noise cancellation circuitry is placed inside the active headset;

FIG. 10 shows a connector using retractable contacts to supply power to the active heads, in accordance with a third embodiment of the present invention in which the noise cancellation circuitry is placed inside the active headset;

FIG. 11 shows an active headset plug configured with a rear power socket, in accordance with a fourth embodiment of the present invention in which the noise cancellation circuitry is placed inside the active headset;

FIG. 12 shows an active headset circuit configured with a power socket in the rear of an active headset plug, in accordance with the fourth embodiment of the present invention in which the noise cancellation circuitry is placed inside the active headset;

FIG. 13 shows a socket using an ultrasonic test tone to determine if supply power is to be provided to the active headset, in accordance with a fifth embodiment of the present invention in which the noise cancellation circuitry is placed inside the active headset;

FIG. 14 shows a phantom powering circuit for providing power to the active headset, in accordance with a sixth embodiment of the present invention in which the noise cancellation circuitry is placed inside the active headset;

FIG. 15 shows a pulse width modulation circuit for providing power to the active headset, in accordance with a seventh embodiment of the present invention in which the noise cancellation circuitry is placed inside the active headset;

FIG. 16a shows a circuit diagram of the output audio signal power produced by the external device being used to power the active headset, in accordance with a eighth embodiment of the present invention in which the noise cancellation circuitry is placed inside the active headset;

FIG. 16b shows a circuit diagram of the output audio signal power produced by the external device being used to power the active headset using a switched-mode power supply within the active headset to boost up the signal voltage level, in accordance with a ninth embodiment of the present invention in which the noise cancellation circuitry is placed inside the active headset;

FIG. 17 shows a linear charging circuit to reduce distortion in the audio playthrough path when the output audio signal power produced by the external device is used to power the active headset, in accordance with a tenth embodiment of the present invention in which the noise cancellation circuitry is placed inside the active headset;

FIG. 18 shows a schematic to eliminate the crosstalk between the earphone and microphone signals associated with using a common ground for the earphones and microphones due to the cable resistance, in accordance with a first embodiment of the present invention in which the noise cancellation circuitry is located at least partially in an external device remote from the active headset;

FIG. 19 shows a diagram illustrating automatically switching the line output socket to serve an auxiliary purpose as an error microphone input socket when an active headset is plugged in, in accordance with a second embodiment of the present invention in which the noise cancellation circuitry is located at least partially in an external device remote from the active headset;

FIGS. 20a, 20b, and 20c show a Pulse Width Modulated (PWM) drive to power the microphone circuitry, in accordance with a third embodiment of the present invention in which the noise cancellation circuitry is located at least partially in an external device remote from the active headset;

FIG. 21 shows a conventional-linear audio signal superimposed upon a positive DC voltage level to power the microphone by filtering off the audio signal and measuring the output of the microphone by means of adding negative-going spikes, in accordance with a fourth embodiment of the present invention in which the noise cancellation circuitry is located at least partially in an external device remote from the active headset;

FIG. 22 shows a bridge circuit used to separate out a microphone signal from the headphone drive, in accordance with a fifth embodiment of the present invention in which the noise cancellation circuitry is located at least partially in an external device remote from the active headset; and

FIG. 23 shows a radio frequency carrier signal used to separate out a microphone signal from the headphone drive, in accordance with a sixth embodiment of the present invention in which the noise cancellation circuitry is located at least partially in an external device remote from the active headset.

FIG. 24a shows a dual mono jack plug connector for use with a mono active headset with electronics separate from the headset and with earphone, microphone and gain adjustment element in the headset. The pin connections to the headset elements are also indicated.

FIG. 24b shows a stereo jack plug connector for use with a mono active headset with electronics separate from the headset and with earphone, microphone and gain adjustment element in the headset. The pin connections to the headset elements are also indicated.

FIG. 25 shows a dual mono plus stereo jack plug connector for use with a mono active headset incorporating a boom microphone assembly with electronics separate from the headset and with earphone, microphones and gain adjustment element in the headset. The pin connections to the headset elements are also indicated.

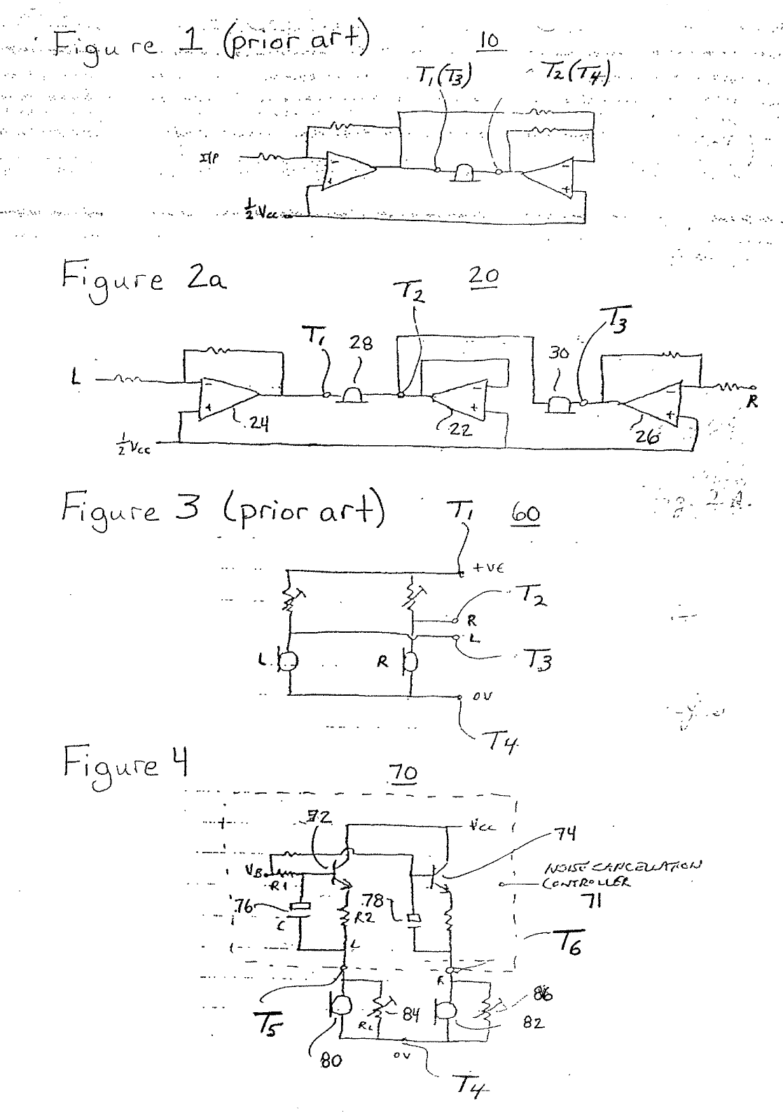

### DESCRIPTION OF THE INVENTION

The present invention describes a noise cancellation system for an active headset in which the number of required connections between noise cancellation circuitry to the active headset is reduced. The same principles of the invention for reducing the number of required connections are applicable to both stereo active headsets as well as mono active headsets, which may be used, for example, in telephone call centers. A mono headset may be employed in order that the wearer is less isolated from the environment. The same advantages still hold in terms of reducing the number of required connections between the headset and electronics. In the case of the mono headset, reducing the number of connections for the active headset also allows a boom microphone to be incorporated into the headset whilst still being able to use a standard connector. Even with a stereo headset with boom microphone, reduction of the necessary connections is still advantageous.

The invention is generally concerned with noise cancellation using a headset including, but not limited to, noise cancellation systems in aircraft and consumer stereo equipment such as Walkmans ®, wherein each user has an associated active headset.

Assuming the gain in the headset is adjustable so that the active headset is usable in any number of applications regardless of manufacturing tolerances in the remote noise cancellation circuits, a minimum of three electrical connections would normally be required for stereo earphones, left, right and common, and a minimum of four electrical connections would normally be required for electret-type microphones, namely left, right, common and power. While in theory the two common lines could be joined together, in practice this creates a significant common impedance between the loudspeakers and the microphones, leading to loss of stability and/or loss of external noise cancellation and/or to commoning of the two channels, especially with low impedance loudspeakers. Moreover, if, as would be preferred in order to prevent roll-off at the lower end of the frequency range of external noise cancellation, the earphones are to be driven by bridge amplifiers, four electrical connections would be required for earphones being thus configured.

There exist several possibilities for reducing to six the number of electrical connections between the active headset and the remote noise cancellation circuitry, thus enabling the use of two stereo jack plugs, or a six pin connector, to provide the necessary connections. Furthermore, assuming the use of transformers is to be avoided, standard “phantom powering” techniques that would require additional circuitry be added to the active headset could be used. Several other possibilities exists for reducing the number of connections between the headset and remote noise cancellation circuit to six.

First, the number of electrical connections required for the earphones is reduced to three by controlling the impedances of the earphones, so that a common contact from amplifiers in the remote noise cancellation circuitry can serve as an input connection to corresponding terminals of the two earphones. While one advantage of bridge amplifier circuitry is lost, i.e. the double swing in amplification, the avoidance of roll off at lower frequencies is achievable by coupling two single-ended operational amplifiers via a third operational amplifier at which the common third terminal is provided, or by grounding through a common terminal one side of both earphones and either supplying the earphones with the noise cancellation signals from single-ended operational amplifiers via suitable capacitances or by connecting two single-ended operational amplifiers between positive and negative power supply voltages. This first arrangement is preferable when only a single power supply is available.

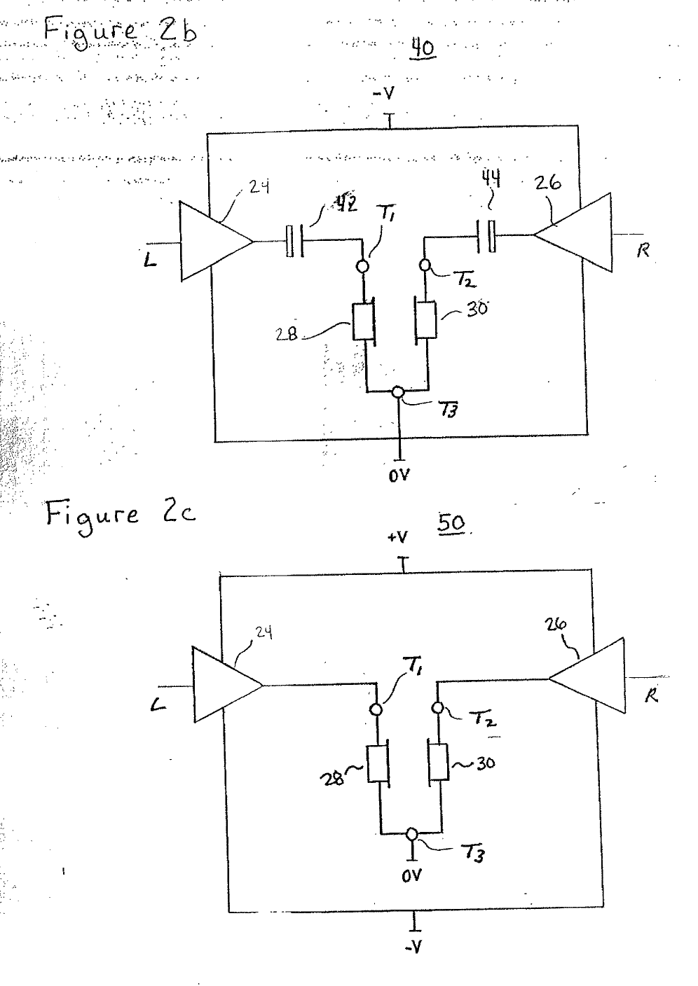

In the arrangement of FIG. 2a, an amplifier circuit is shown in which the impedances of the earphones are controlled, reducing the number of terminal connections from four to three. A third operational amplifier 22 is connected between two single ended operational amplifiers 24, 26, one for each earphone. L and R indicate the left and right noise cancellation signals input to the respective amplifiers 24, 26. T₁, T₂ and T₃ are the three connection points between the headphones 28, 30 and the remote noise cancellation circuitry.

FIGS. 2b and 2c show two alternative circuits which may be used in place of the circuit of FIG. 2a. In each case, one side of the two earphones 28, 30 is grounded. In FIG. 2b, the noise cancellation signals are fed to the earphones via suitable capacitances 42, 44. The arrangement of FIG. 2c requires the availability of a negative power supply (−V). The three connection points between the headphones 28, 30 and the remote noise cancellation circuitry are again indicated as T₁, T₂ and T₃.

In the case of a mono headset, it is of course not possible to make use of a common connection between the earphones—there is only one earphone and that requires two connections.

Second, the number of connections required for the earphones may be reduced by configuring bootstrapped emitter-follower transistors in the remote control circuitry to not only act as current sources at audio frequencies but also to provide correct bias voltage to the microphones, whereby a common contact can serve as an input connection to corresponding terminals of the two microphones in the earpiece. Alternatively, operational amplifiers may be used instead of emitter-follower transistors.

FIG. 4a is a circuit diagram for a circuit in the remote noise cancellation controller 71 for handling the signals from the microphones in the headset in accordance with the invention, wherein emitter follower transistors 72, 74, replaceable by operational amplifiers if desired, act as current sources at audio frequencies by virtue of bootstrapping capacitors 76, 78, while simultaneously providing the correct bias voltages to the microphones 80, 82. The circuitry of remote noise cancellation controller 71 is inside the dashed lines of FIG. 4. The sensitivity of each microphone remains fully adjustable by potentiometers 84, 86, and only three terminal connections T₄, T₅ and T₆ are necessary between the microphones and the remote noise cancellation controller 71. V_{cc} indicates the rail voltage, V_{B}, a bias voltage.

FIG. 4b shows the circuitry that would be required for the mono version of such a headset. Only two terminal connections T₄ and T₅ are necessary between the microphone and the remote noise cancellation controller 71.

The only minor disadvantages of the arrangements above-described are an increase in current drawn and a slightly reduced bandwidth in noise cancellation in order to maintain stability.

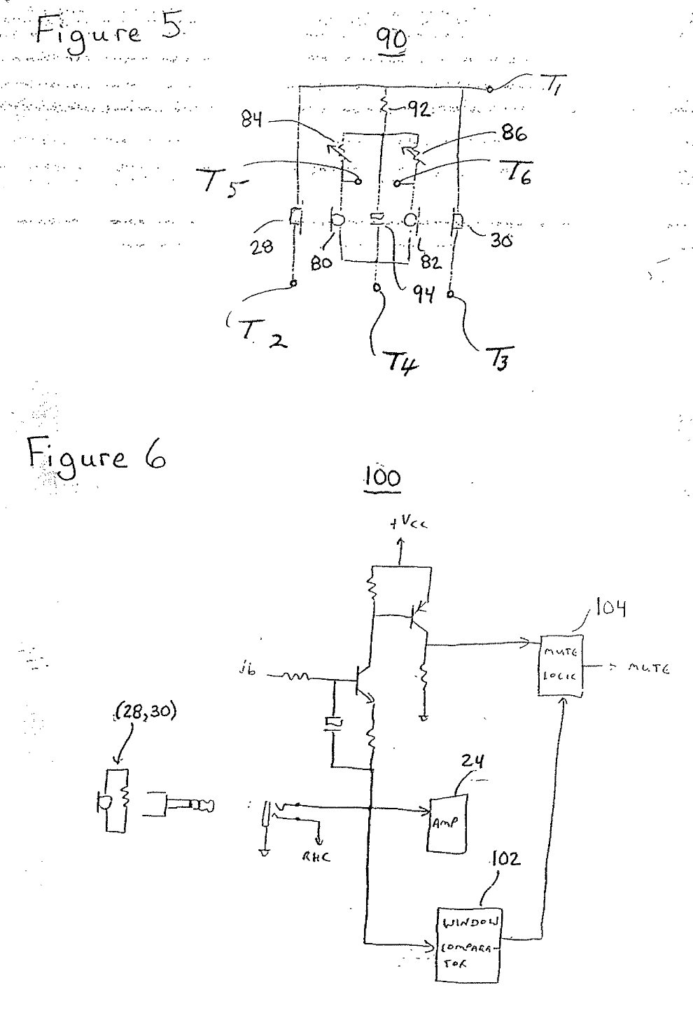

FIG. 5a shows an alternative arrangement in accordance with the present invention. This arrangement is based on the concept of freeing the common line of the earphones from any requirement to be at ground potential. The output from the power amplifier in the noise cancellation circuitry is capacitatively coupled to remove DC offset. The common earphone connection is then made at T₁. The common line for the earphones 28, 30 is then connected to the power supply line to the microphones 80, 82, so that the common line assumes a DC potential with respect to the microphone ground. A few inexpensive additional components are required in the headset to filter off any signal voltage in the supply. As indicated, a resistor 92 and a capacitor 94 would be required, and possibly in some cases a zener diode. These elements could be located on the printed circuit board which carries the sensitivity potentiometers 84, 86.

The overall arrangement would necessitate only six terminal connections T₁, T₂, T₃, T₄, T₅ and T₆ between the headset and the noise cancellation circuitry, as indicated in FIG. 5. The bootstrapping circuit of FIG. 4 is no longer required.

FIG. 5b shows the same approach used with a mono headset, but instead of the common connection between the earphones being tied to terminal T₁ only one of the terminals of the mono earphone is tied to it instead.

Once the number of electrical connections between the active headset and the remote noise cancellation circuitry has been reduced from eight to six, compatibility with existing stereo jacks or six pin connectors is assured. The noise cancellation circuitry may then be provided within an external device and the external device provided with a six contact socket or two stereo jack plug sockets. In the latter case, preferably one socket provides for three input connections to the gain adjustable microphones in the earpieces and one stereo jack plug socket provides three input connections to the earphones in the earpieces and thus is alternatively usable to plug in a conventional passive headset.

The arrangements above-described enable the use of two stereo jack plugs to connect an active headset to remote noise cancellation circuitry. Thus, in an aircraft, the noise cancellation circuitry can be located in the arm rest of a seat, which arm rest is provided with two stereo jack plug sockets for receiving two stereo jack plugs provided on the active headset.

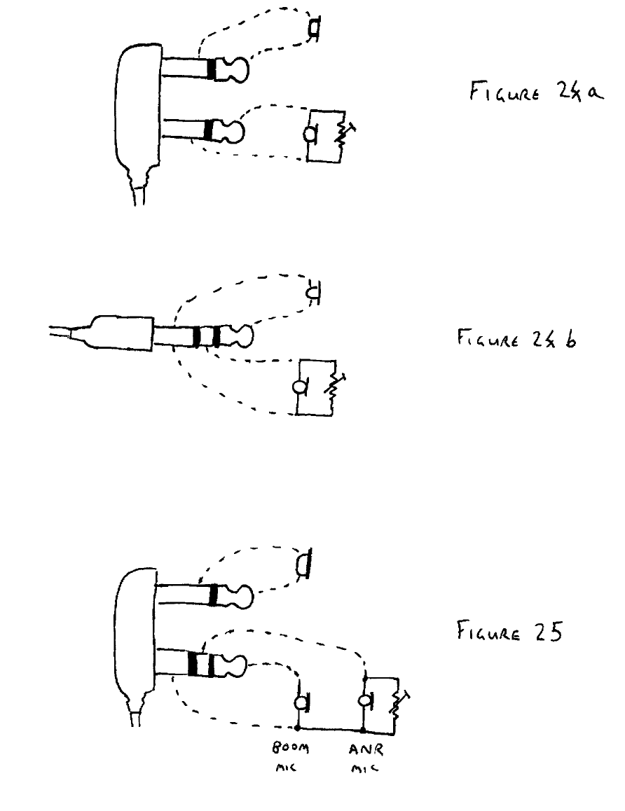

In the case of a mono headset, the techniques described above can be used to advantage also as shown in FIGS. 24a and 24b. FIG. 24a illustrates how a dual mono jack plug could be used to provide for connection between the headset and remote electronics. FIG. 24b shows the connections when using a single stereo jack plug. In this case one of the microphone connections and one of the earphone connections are commoned. This could lead to crosstalk problems unless the common resistance is low.

FIG. 25 shows a possible connection arrangement if a boom microphone is incorporated in the active headset, using a mono and stereo jack plug.

Another problem which can arise when the noise cancellation circuitry is remote from the headset is that because power is necessary for the microphones, there is a DC offset at the microphone terminals, larger than the signal voltages, that can give rise to a large transient in the headphones when plugging in and unplugging the stereo jack plugs (or six pin connector). This can be overcome by the provision of a transient detector in the noise cancellation circuitry. For example, the transient detector may comprise a window comparator and a mute logic circuit for muting any output to the earphones that exceeds a predetermined amplitude level.

In order to avoid a “plop” noise in the ear when the stereo jack plugs are plugged in and unplugged when the headset is being worn, a transient detector and suppressor may be provided in the noise cancellation circuitry. As shown in FIG. 6, in which the noise cancellation circuitry includes a window comparator 102 and a mute logic circuit 104 for muting signals to the earphones 28, 30 if the amplitudes of those signals exceeds a predetermined value. The circuitry shown is that for one earphone, for example the left earphone. RHC indicates the connection leading to a similar circuit for the right earphone. As before V_{cc} indicates the rail voltage (power supply) and V_{B}, a bias voltage.

The removal of the plug-in transient is relatively straightforward. Before the headset is plugged in, the microphone connection point is left open circuit and so no current flows in the emitter follower transistor, and the bootstrapping capacitor is discharged. The voltage at the microphone input is at V_{B} under these conditions. When the microphone makes connection, the voltage is initially pulled down to ground since the bootstrapping capacitor cannot instantaneously charge up to allow current to flow in the transistor. This transient would normally get through to the earphone. Gradually the capacitor charges up and re-establishes the correct voltage on the microphone and the correct current in the transistor. The mute circuit keeps the power amplifier disabled until it detects that the current has passed a predetermined threshold, indicating that the microphone is now in circuit, and then only after a set delay is the power amplifier enabled. In this way the transient has disappeared before the power amplifier becomes active.

When the headset is unplugged there is a transient whose direction depends on the geometry of the plug and socket. If a clean break occurs then the transient is positive due to the transistor attempting to maintain the previous current flow until the bootstrapping capacitor discharges. If, however, the plug momentarily shorts the microphone input to ground as it is withdrawn, the transient is negative. These transients have a steep edge which is fed through to a window comparator 102 which detects whether the comparison result exceeds a predetermined positive or negative threshold. If the thresholds are exceeded, the power amplifier is rapidly muted. In this way the circuit can differentiate between disconnection transients and the normal signal voltages that are present at the input.

This transient elimination circuitry is also applicable to the mono headset case, in which the transient detection circuit may be connected to the microphone in the headset used for active noise reduction. Although a stereo jack plug is shown in FIG. 6, this is simply by way of illustration—the operation of the transient elimination circuitry is independent of the style of connector.

It should be noted that if the circuit of FIG. 5, rather than the bootstrapping circuit of FIG. 4, were used in conjunction with the transient detector circuit of FIG. 6, then the portion of FIG. 6 applicable to plugging-in transients could be removed.

Alternately, it is possible to provide a decoupling capacitor across the power supply to the microphone in each earpiece, with a DC blocking capacitor between the microphone output and the signal lines. This solution simplifies the remote noise cancellation circuitry in return for the introduction of a few inexpensive components into the active headset and boot strapping circuitry or muting circuitry becomes unnecessary.

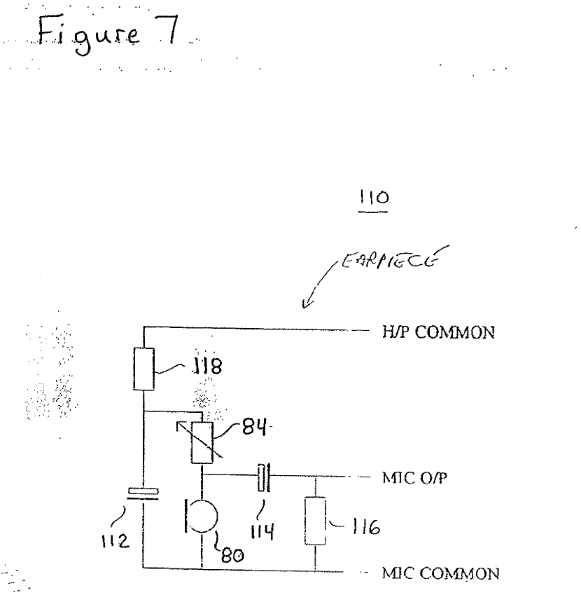

As shown in the transient elimination circuit of FIG. 7 for one earphone 28, the addition of decoupling capacitor 112 and AC coupling capacitor 114 in the headset will overcome the plugging in/unplugging problem. When the headset is plugged into or unplugged, the power applied to the microphone circuit charges or discharges the microphone power supply decoupling capacitor 112 only slowly through the series resistor 118. The change in voltage on the microphone output is too slow to pass through the AC coupling capacitor 114 and so does not cause a transient on the input to the following circuitry. The use of a transient detector in the noise cancellation circuitry, whether of the kind shown in FIG. 6 or otherwise, is thereby avoided, but some inexpensive components are added to the headset, as indicated by the capacitors 112, 114 already referred to, and resistors 116 and 118. These additional components, while being required in the headset for each earphone, simplify the noise cancellation circuitry in that the bootstrapping circuitry of FIG. 4 and the muting circuitry of FIG. 5 are no longer necessary. It is further noted that the headphone common and microphone common shown in FIG. 7 are terminals of the circuitry; in the case of a mono headset, these would be referred to as “H/P TERMINAL” and “MIC TERMINAL,” for instance.

When the active noise cancellation circuitry is placed inside the active headset, power must be provided to the headset for the active noise cancellation circuitry. The power for the active headset could be obtained from the external device that is responsible for generating an audio signal to the headset. Several means are available to provide power to the active headset from the external device: the number of contacts on the connector may be increased to supply power to the active headset; a connector using a single supply rail may be used to supply power to the active headset; retractable contacts on the plug may be used to supply power to the active headset; sockets on the external device, such as the external power socket or the line out socket for low level audio signals, may be used to power the active headset; phantom powering may be used to power the active headset; a pulse width modulation (PWM) amplifier may be used to power the active headset; or the output audio signal power produced by the external device may be used to power the active headset.

Of the above, the simplest method is to modify the connector to increase the number of contacts. Jack plugs and sockets exist for telephony applications which have additional connection rings over those used for stereo applications. This approach could therefore be easily used for supplying power to the headset.

One technique to increase the number of contacts is to implement an extra contact beyond the distance that a stereo plug would extend into the socket so that a stereo plug would not make electrical contact with it. This extra contact carries the power needed to power the noise cancellation circuitry, the supply earth being combined with the signal earth. Compatibility with a normal non-active headset is retained since a normal non-active headset plug would not extend far enough into the socket to connect with the power contact.

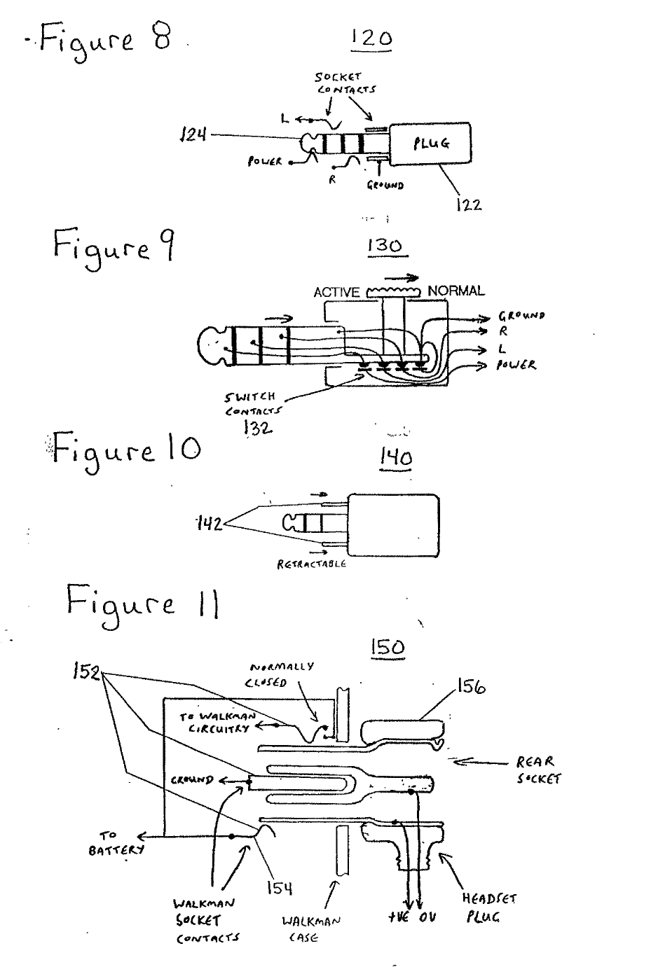

Referring to FIG. 8, a connector 122 using a single supply rail to supply power to the active headset is shown. Implementing a single supply rail for the headset requires an extra contact 124 beyond the distance that a stereo plug would extend into the socket. This extra contact carries the power, the supply earth being combined with the signal earth. Compatibility with a normal non-active headset is retained since a if normal non-active headset plug would not extend far enough into the socket to connect with the power contact. The active headset would use an extra long plug with an extra contact to pick up the power.

The active headset also retains compatibility with normal headphone sockets since normal headphone sockets usually are open at the end or longer than the plug length. The extra length of the plug would thus extend harmlessly beyond the normal contacts. The active headset circuit passes an audio signal straight through to the headphone when there is no power supply present to activate the cancellation circuitry. If the length of the plug does prove to be a problem with some sockets, then an adapter could be supplied with the active headset so that it can be plugged into normal sockets.

A second technique to increase the number of contacts is to implement a connector using switch contacts to supply power to the active headset. As shown in FIG. 9, a connector uses switch contacts 132 to supply power to the active headset.

As the switch is moved between “normal” and “active” positions the contact end of the plug extends and at the same time switch contacts within the plug housing reconfigure the connections to take care of routing the signals correctly.

These techniques could be extended if dual rails are required, but the extra length needed to accommodate two extra contacts could be problematic unless the switchable plug approach was used to ensure compatibility.

A third technique to increase the number of contacts is to implement a connector using a retractable contacts to supply power to the active headset. Compatibility with a normal non-active headset socket is retained since the pins either retract automatically when the plug is inserted into a normal non-active headset socket, or can be retracted manually.

Alternatively, the plug could be made into an alterable format as shown in FIG. 10, a connector using a retractable contacts to supply power to the active headset. Retractable thin pins 142 are added around the periphery of a standard jack plug. These pins are small enough so as to cause only a minimal increase on the socket diameter. The plug is designed such that the pins are retractable in a non-active mode. Compatibility with a normal non-active headset socket is retained since the pins either retract automatically when the plug is inserted into a normal non-active headset socket, or can be retracted manually.

An alternative to expanding the number of contacts on the headphone socket is to make use of other sockets that already exist on the external device. If the external device is optionally battery-powered and has an external power socket such as a Walkman ®, for instance, then the external power socket may be used. The external power socket is replaced with a dual function socket that disconnects the batteries if a normal plug is inserted, but connects the batteries if a special active headset plug is used thereby powering the noise cancellation circuitry. Alternatively, the active headset plug may be configured with a power socket in the rear of the headset plug so that the external device could still be used with an external power supply when the active headset is being used.

The techniques illustrated in FIGS. 8 through 10 could also be used in the case of a mono headset with boom microphone, such as when used with a computer for voice recognition purposes, by replacing the connection to the right-hand channel of the headset with a connection to the boom microphone. The connection previously made to the left channel is now the signal input for the mono signal to the earphone(s).

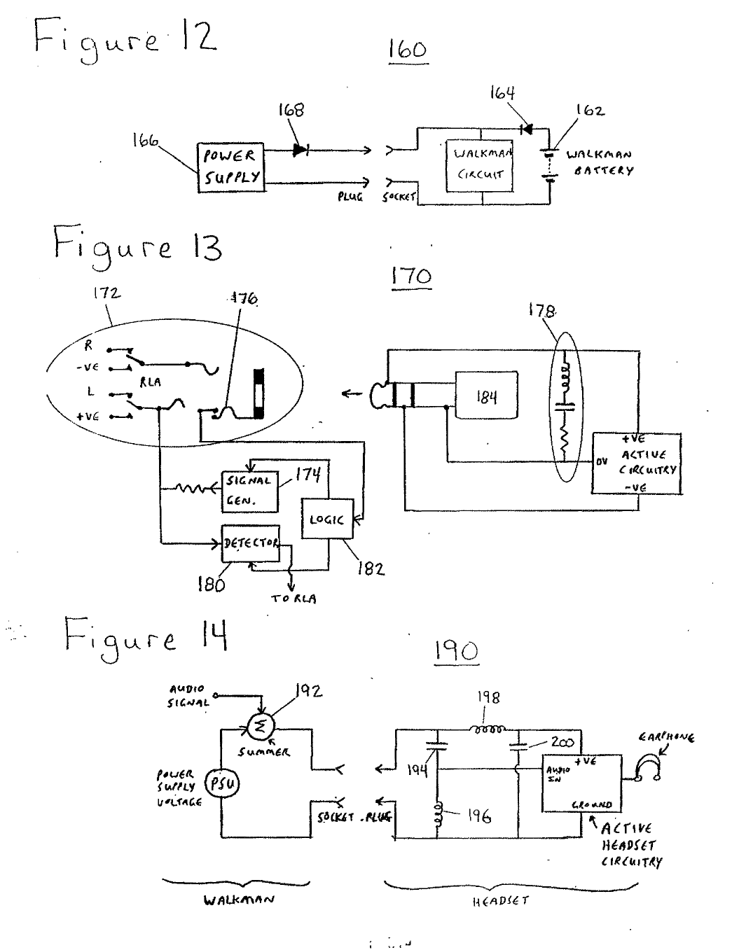

Normally the external power socket is designed to act as a power input socket and therefore disconnects the batteries when a plug is inserted in order to avoid the power supply damaging the batteries. As shown in FIG. 11, the external power socket is replaced with a dual function socket 152 that disconnects the batteries if a normal plug is inserted, but connects the batteries if a special active headset plug is used. This may be accomplished by extending the length of the external power socket and adding an extra contact 154 that is always connected to the battery. Alternatively, the external power socket may be implemented with the battery disconnect function being performed electronically instead of with a physical battery disconnect switch. In this configuration (FIG. 12), the battery 162 is connected to the external power socket and the external device circuit via a diode 164, and likewise the external power supply 166 is connected via a diode 168 to the power supply plug. In this way, whichever voltage is the larger will determine which power source supplies the circuitry. Thus, no current will be able to flow into the battery to cause damage.

The active headset plug 156 may be configured with a power socket in the rear so that the external device could still be used with an external power supply when the active headset is being used as shown in FIGS. 11. In this configuration, the active headset plug would connect directly to the headset power supply and the external power socket on the external device.

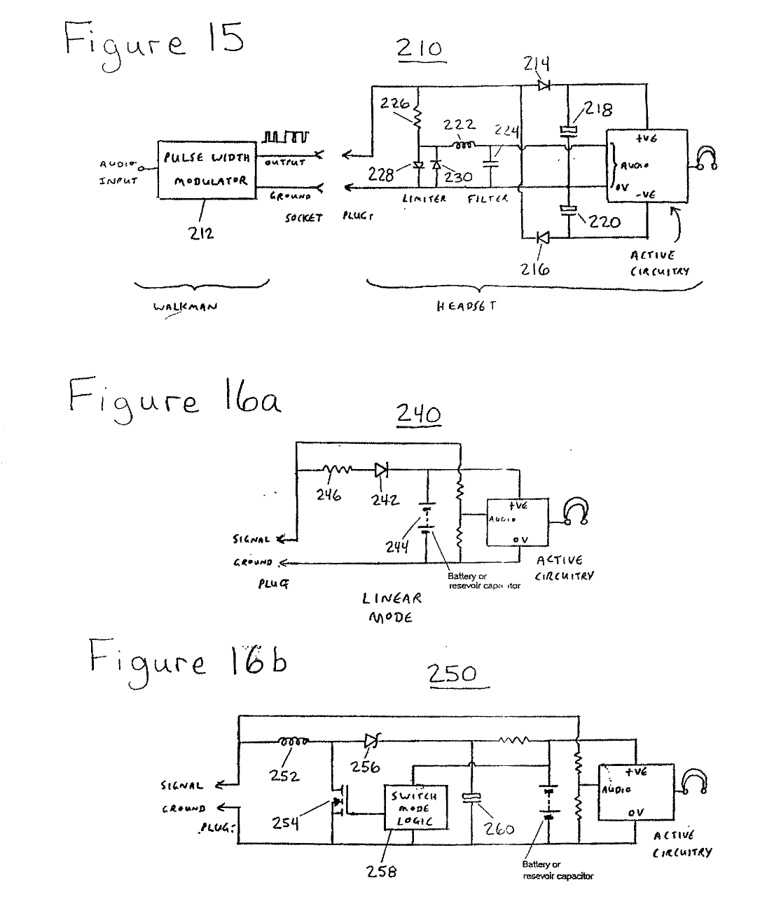

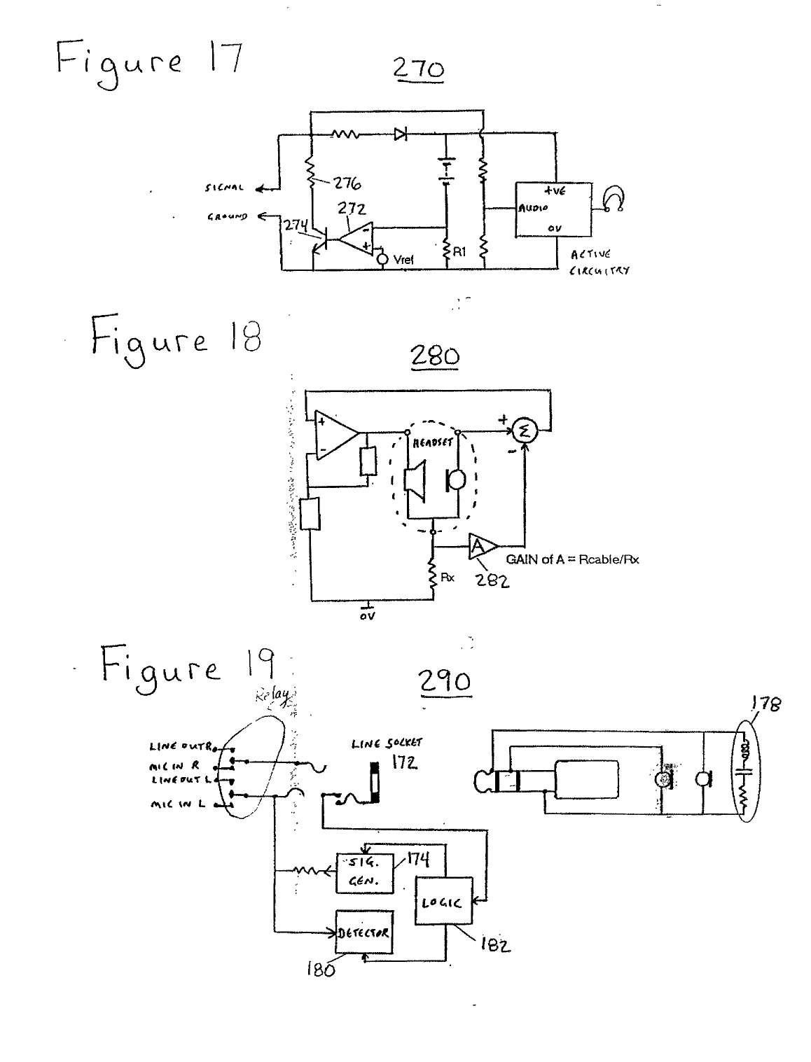

Other sockets on the external device may be used to power the active headset. If the external device has a line-out socket for low level audio signals, then it may be used to power the active headset. The function of the line-out socket is electronically switched over to supply power instead of an audio signal when an active headset is plugged into the socket. As shown in FIG. 13, a socket uses an ultrasonic test tone to determine if supply power is to be provided to the active headset. In this configuration, the function of the line out socket 172 is electronically switched over to supplying power instead of an audio signal when an active headset is plugged into the socket. This may be implemented by a sensing system that superimposes an ultrasonic test tone from generator 174 onto the audio output when a headset plug 184 is first inserted. An ultrasonic test tone would be generated whenever the normally closed earth contact switch 176 on the line out socket 172 is opened due to insertion of the headset plug. In this configuration, the active headset is arranged to present a specific impedance to the ultrasonic test tone signal by means of network 178, whereupon, controlled by detector 180 and logic 182, the function is then switched over to supplying power to the active headset.

Phantom powering techniques may also be used to power the active headset using the same wires as are used for the audio signal thus requiring no additional connections on the headset plug and socket. The power supply voltage is supplied directly to the headset socket with the audio signal voltage superimposed on top of the power supply voltage by means of a summer. The power supply voltage is then lowpass filtered to strip away the audio before being fed to the headset circuit power supply system. Phantom powering is a technique that is used in music and PA systems for powering microphones using the same wires as are used for the audio signal. This technique may be used for powering active headsets thus requiring no additional connections on the headset plug and socket.

In the phantom powering circuit of FIG. 14, the power supply voltage is supplied directly to the headset socket with the audio signal voltage superimposed on top of the power supply voltage by means of summer 192. The power supply lines on the active headset are high pass coupled into the headset audio input with capacitor 194 and inductor 196, thus blocking the DC power and recovering the audio. The power supply voltage is then low pass filtered by inductor 198 and capacitor 200 to strip away the audio before being fed to the headset circuit power supply system.

To retain compatibility with normal headsets, a means must be employed to detect a normal headset so that the power supply voltage can be removed from the socket to prevent damage to the normal headset. The technique described above of implementing a ultrasonic test tone signal may be used to remove the power supply voltage whereby the active headset is arranged to present a specific impedance to the ultrasonic test tone signal to determine if the power supply voltage should be supplied.

Yet another alternative is to use a pulse width modulation (PWM) amplifier to power the active headset. The PWM amplifier produces a waveform that has a square wave with a frequency that is much higher than the highest desired audio frequency. The mark-to-space ratio, the ratio of the time the square wave is at a high positive voltage to the time it is at a low or high negative voltage, is made proportional to the amplitude of the audio signal. The mark-to-space ratio is unity in the absence of modulation. The average strength of the waveform is proportional to the modulating signal, such that when the high frequency square wave carrier is filtered off, the audio is recovered intact. This happens naturally in a conventional headset by a combination of the inherent inductance of the voice coil and the acoustic roll-off of the earphone. Thus, compatibility is retained with conventional headphones. The PWM output signal may be automatically disabled upon detection of a normal headset.

FIG. 15 shows such a pulse width modulation circuit. The pulse width modulation amplifier 212 produces a waveform that has a square wave with a frequency that is much higher than the highest desired audio frequency. When an active headset is plugged in, the squarewave is rectified and filtered using diodes 214 and 216 and capacitors 218 and 220 to produce the power to drive the circuitry. The audio is recovered by low pass filtering the incoming squarewave with inductor 222 and capacitor 224 and feeding it to the audio input. One potential problem with this technique is that cable resistance may cause the recovered audio signal to become distorted due to the current drawn by the active headset circuit causing voltage drops across the cable resistance. This may be rectified by passing the squarewave through a limiter, formed in this instance by resistor 226 and diodes 228 and 230, before reaching the low pass filter to eliminate the distortion.

The PWM output signal may be automatically disabled upon detection of a normal headset. This is desirable to reduce the interference to other equipment caused by the high frequency nature of the squarewave causing radio frequency radiation from an unscreened headphone cable. The rectification and smoothing process within the active headset causes a different current waveform profile than a normal headset. This information may be used either to revert to a conventional power amplifier for the normal headset or to switch in a low pass filter that removes the squarewave but leaves the audio content intact. Thus, the presence of an active headset may be inferred from the current drawn from the power supply.

The output audio signal power produced by the external device may also be used to power the active headset. If the external device produces an output audio signal power sufficient to power an active headset, then it may be used to power the active headset. Deriving the power for the active circuitry from the output audio signal would not require any modification to the connectors or changes in the external device circuitry, other than to ensure that the available signal voltage of the output audio signal is sufficient to additionally power the active headset. The output audio signal from the external device is turned up to the fullest volume commensurate with the power amplifier not clipping. The audio signal is then rectified and at audio signal peaks the output audio signal charges a reservoir capacitor. The power for the active headset is derived from the audio signal at audio signal peaks and from the reservoir capacitor otherwise. A switched-mode power supply may be added within the active headset to boost the signal voltage level of the output audio signal so that the battery or the reservoir capacitor can be charged even at low audio signal levels. The audio signal input to the noise cancellation system would be derived from an attenuated version of the power signal used to activate the headset circuitry. A dummy load may be switched across the signal line at low signal voltage and removed at higher voltages to obliterate distortion in the audio path introduced by any significant resistance in the headset cable.

The output audio signal power produced by the external device may be used to power the active headset as shown in FIGS. 16a and 16b. If the external device produces an output audio signal power sufficient to power an active headset, then it may be used to power the active headset. For instance, typically the output power that can be produced from a Walkman® is higher than that needed to drive a well designed active headset. Deriving the power for the active circuitry from the output audio signal would not require any modification to the connectors or changes in the external device circuitry, other than to ensure the available signal voltage was high enough for the intended purpose.

Referring to FIG. 16a, the audio signal from the external device is turned up to the fullest volume commensurate with the power amplifier not clipping. Diode 242 in the active headset rectifies the audio signal and at audio signal peaks the audio signal is used to charge a reservoir capacitor 244 through current limiting resistor 246. The power for the active headset is derived from the audio signal at audio signal peaks and from the reservoir capacitor otherwise. To prevent the likelihood that the circuit would run out of power during quiet passages in the music, the reservoir capacitor may be replaced with a small rechargeable battery which would operate like a very large value storage capacitor. Provided the external device power amplifier has a larger voltage output capability than that necessary to operate the active headset, then the battery can be charged even with the average value of the audio signal rather than just the peaks.

As shown in FIG. 16b, a switched-mode power supply formed by elements 252-260 may be added within the active headset to boost up the signal voltage level so that the battery or reservoir capacitor can be charged even with low audio signal levels. The audio signal input to the system would be derived from an attenuated version of the power signal used to activate the headset circuitry.

Any significant resistance in the headset cable may introduce distortion into the audio playthrough path when the output audio signal power produced by the external device is used to power the active headset. This arises because the current drawn from the audio power is not a linear function of level. At low signal levels there is insufficient voltage present to charge the storage system, even when using a switched-mode power supply, and thus no current will be drawn from the audio. Since it is the voltage at the end of the cable that is bled off to provide the audio playthrough component, the voltage drop across the cable will therefore only occur at the higher signal voltages thereby producing the distortion. To alleviate this problem, a dummy load, may be switched across the signal line at low signal voltage and removed at higher voltages. Thus, the total load resistance presented across the cable is signal independent and the distortion is reduced. Such a scheme for a linear charging circuit is shown in FIG. 17. When the current through the battery falls below a preset level of Vref/R1, the comparator 272 switches on transistor 274 to place resistor 276 across the signal line. The value of resistor 276 is chosen to maintain the same input resistance at low signal levels as exists at high levels.

A related solution that also uses the output audio signal power borrows from the PWM approach previously discussed. A high-frequency pulse train superimposed on the power amplifier output charges up the reservoir capacitor. The pulses would be of full amplitude at an inaudibly high frequency. The audio signal would be arranged to be always lower in amplitude than the pulses. By keeping the pulse width narrow, the interference is kept to a minimum even with unscreened cables, but there would still be sufficient pulse width to replenish the reservoir capacitor charge. This scheme would reduce the effects of cable resistance on audio signal distortion.

When at least some of the active noise cancellation circuitry is remotely located inside an external device, such as inside a Walkman® or other electrical connections must be made to the headset for the earphones and the error sensing microphones. This requires potentially more connections and involves additional problems due to the low signal level from the error microphones. The simplest approach is to increase the number of connections on the socket. However, it is possible and desirable, by putting some circuitry inside the active headset, to reduce the required number of electrical connections to three and at the same time maintain compatibility with normal passive headsets. These various techniques are described below.

The jack plug or a socket on the external device may be used as contacts to the active headset while still retaining compatibility with normal passive headsets. If an additional socket is not available on the external device then one of the sockets already present on the external device may be used to serve an auxiliary purpose when an active headset is plugged in.

It has been described above how the jack plug and socket can be altered to accommodate an extra contact while still retaining compatibility with normal headsets. The minimum number of contacts needed for a straightforward connection regime to the headset is five if the microphones and earphone all share a common ground. This requires two extra contacts on the jack plug resulting in a jack plug that is too long to fit into a standard stereo jack socket. The switchable jack plug approach shown in FIG. 9 would still be useable. In addition, extra peripheral pins could also be used for the microphone connections in the same manner as outlined for transferring power to an integrated active headset.

A potential problem associated with using a common ground for the earphones and microphones is crosstalk between the earphone and microphone signals due to the cable resistance. The crosstalk between the earphone and microphone signals due to the cable resistance may be eliminated if the cable resistance is known as is shown in FIG. 18. A resistor Rx is placed between the cable common ground wire and circuit ground. The voltage drop across this resistor is sensed and buffered by amplifier 282 and subtracted from the microphone output, thus eliminating the crosstalk.

Other sockets on the external device may be used as contacts to the active headset as is shown in FIG. 19. As described above, if an additional socket is not available on the external device then one of the sockets already present on the external device may be used to serve an auxiliary purpose when an active headset is plugged in. For instance, the line output socket 172 may double as the error microphone input socket. In this configuration, the line output socket is switched automatically by Relay of FIG. 19 by interrogating the headphone output socket with an ultrasonic tone in the same manner as described for FIG. 13. The active headset presents a pre-determined impedance at this frequency causing the mode of the line socket to be switched over.

By including some of the signal conditioning circuitry within the headset, it is possible to reduce the required number of contacts to three even when the active circuitry is contained within the external device. This technique is most applicable for higher performing active headsets where a much more complex equalizer might be needed. In this instance, it would be preferable to keep most of the electronics inside the external device. The various methods for combining the earphone and microphone connections are described below.

The drive signal to the headset earphone may be converted into a Pulse Width Modulated (PWM) drive signal to power the microphone circuitry. Power for the microphone can be derived in a similar manner to the technique described above to supply an integrated active headset, without corrupting the drive for the earphone. Compatibility is retained with a normal headset because of the bi-directional nature of the PWM drive signal. Since a conventional headset lacks the internal diodes, the PWM is presented to the conventional headset without a DC offset and the carrier signal is filtered out by a combination of the earphone inductance, the natural high frequency roll-off of the headphone frequency response, and the limited hearing range of the ear. Alternatively, the output may be switched from the PWM amplifier to a conventional linear amplifier.

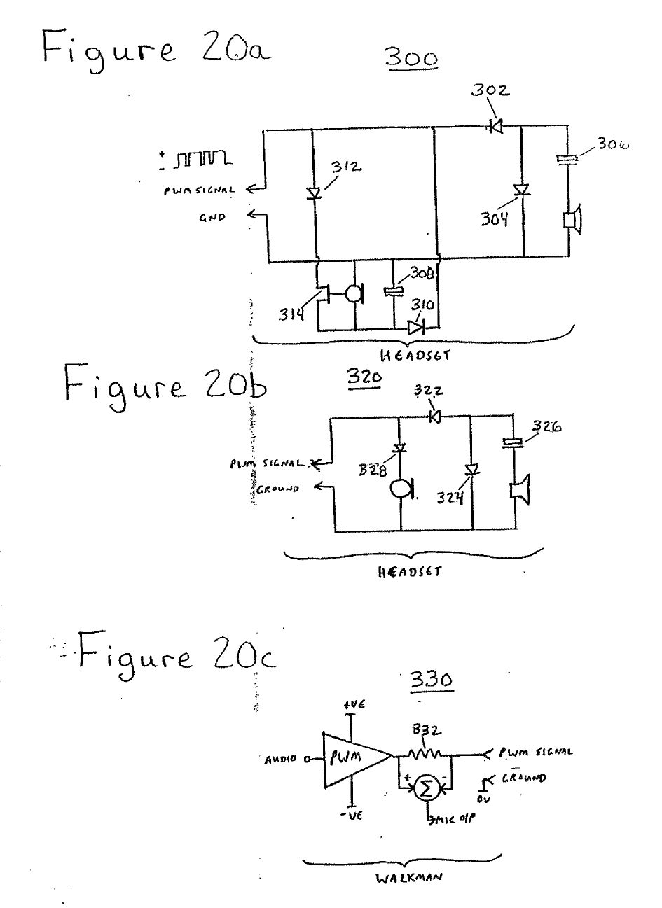

The drive signal to the headset earphone may be converted into a Pulse Width Modulated (PWM) drive to power the microphone circuitry as shown in the simplified circuits in FIGS. 20a, 20b, and 20c. The constant height of the square wave signal, combined with the fact that even when the modulating audio signal is at zero the square wave is still present, is ideal. Power for the microphone can be derived in a similar manner to the technique described above to supply an integrated active headset, without corrupting the drive for the earphone.

In FIG. 20a a bi-directional PWM square wave is connected to the earphone through diodes 302 and 304 such that the earphone is only connected in circuit when the square wave is at the negative voltage level. This causes a DC offset on the earphone, but will not otherwise affect the recovered audio, and the DC offset can be removed by capacitor coupling the earphone with capacitor 306. When the square wave is at a negative voltage it is also used to charge a reservoir capacitor 308 via diode 310 to power the error microphone. Only minimal power is necessary to charge the reservoir and thus does not appreciably affect the signal sent to the earphone. When the square wave is at a positive voltage it is isolated from the earphone but connected by means of a further diode 312 to the microphone output circuit 314 such that the microphone signal level can be read during this period by means of a current sensing resistor 332 in the output drive line in the external device, as shown in FIG. 20c. It is also possible to dispense with the need to continuously power the microphone and only connect it into circuit for the periods that the square wave is positive thus saving components in the headset as in FIG. 20b. Diodes 322 and 324 and capacitor 326 in FIG. 20b perform the same purpose as elements 302, 304 and 306 in FIG. 20a. Diode 328 only connects the microphone in circuit during the positive going period of the squarewave.

Compatibility is retained with a normal headset because of the bi-directional nature of the PWM drive signal. Since a conventional headset lacks the internal diodes, the PWM is presented to the conventional headset without a DC offset and the carrier signal is filtered out by a combination of the earphone inductance, the natural high frequency roll-off of the headphone frequency response, and the limited hearing range of the ear.

The symmetry of the current drawn during positive and negative half cycles of the squarewave at times when the mark to space ratio is unity is monitored to detect when a normal headset is plugged in to alter the equalization applied to the audio signal. When a normal headset is plugged in, the current flow is symmetrical, but when an active headset is plugged in, the current flow is not symmetrical. As well as switching the equalization, an output filter may be switched into place when a normal headset is detected in order to reduce radio frequency radiation. Alternatively, the output may be switched from the PWM amplifier to a conventional linear amp.

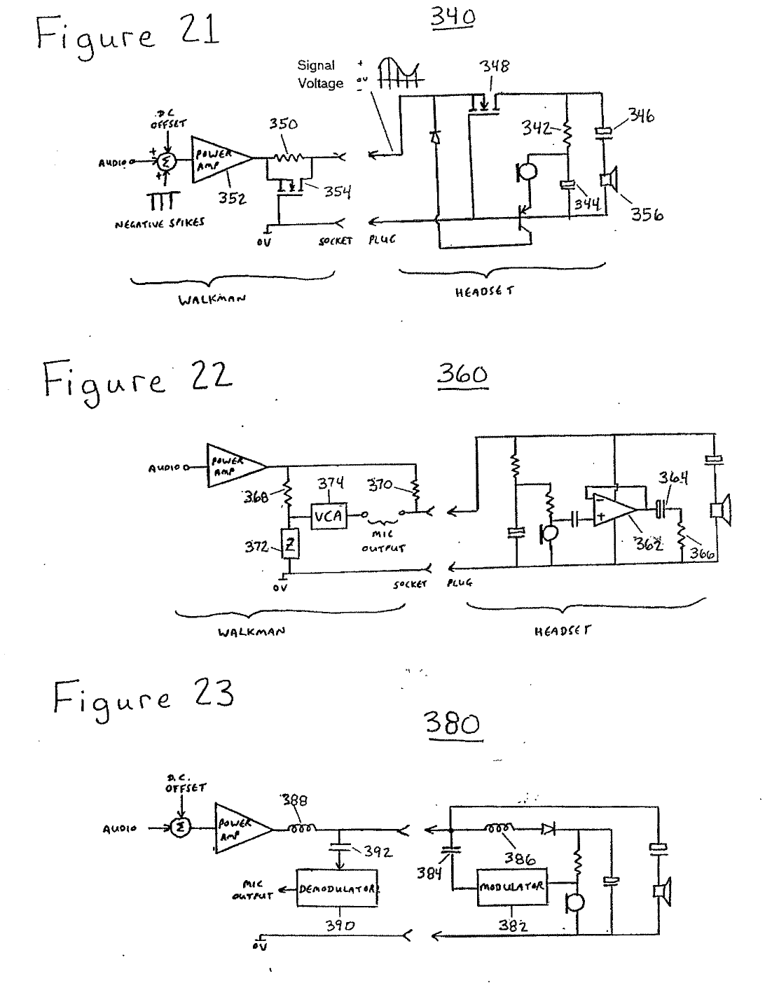

Another alternative uses a conventional analog output by superimposing a conventional-linear audio signal upon a positive DC voltage level configured such that the combined voltage level never goes below ground as shown in FIG. 21. The DC level is used to power the microphone by filtering off the audio signal with resistor 342 and capacitor 344. When ever the voltage level is above ground, P-channel FET 348 is held on so that the signal is connected through to the earphone. As before the earphone can be capacitor coupled within the headset to remove the DC offset.

To read the microphone output signal, very short duration high frequency negative-going spikes are superimposed upon the drive signal. For the period of the negative spike, the earphone is temporarily disconnected from the power amp as Field Effect Transistor (FET) 348 turns off and the signal line is instead connected to the microphone. Since the spikes are a very high frequency they are not audible in the earphone. A current sensing resistor 350 is used in series with the power amp 352 with FET 354 used to short it out when the signal level is positive. In this way a higher value can be used for resistor 350 to produce a higher microphone output signal without causing signal loss to the earphone 356.

To maintain compatibility with a normal headset in this configuration, the DC offset applied to the headset must be eliminated when a normal headset is plugged in. This may be accomplished by increasing the DC voltage slowly from zero whenever a headset is connected. The DC voltage is increased to its full level only if the presence of a microphone is detected, by measuring whether current is drawn during the period of the negative-going spikes. Otherwise, the DC voltage is reduced to zero and the negative spikes removed. The audio equalization is switched as appropriate. Alternatively, the ultrasonic measuring system described in relation to FIG. 19 could be employed to detect the active headset.

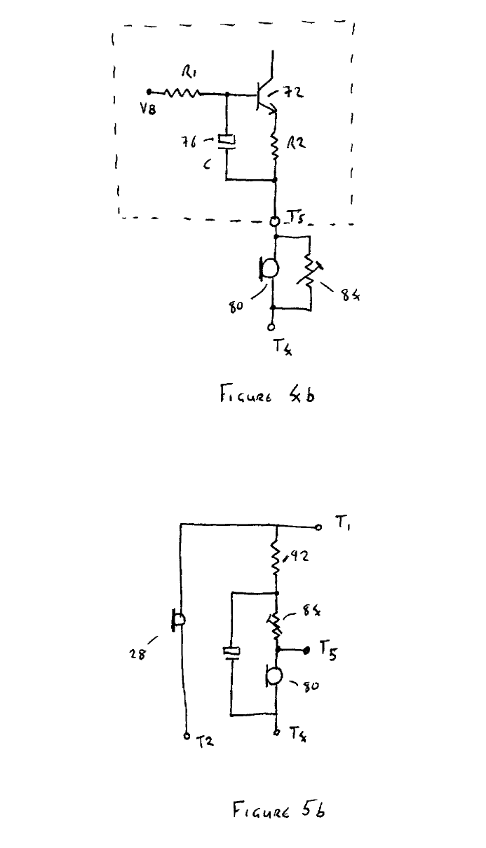

Alternatively, a bridge circuit may be used to separate a microphone signal from the headphone drive signal as shown in FIG. 22. As with the Pulse Width Modulated drive technique describe above, a DC offset is added to the earphone drive signal in order to power the microphone circuitry. The earphone is then capacitor-coupled within the headset to remove the DC offset. The microphone output is converted to a signal dependent current source by op-amp 362, capacitor 364 and resistor 366 and is connected in parallel with the earphone. In the external device, the headset signal is connected into a bridge circuit formed by resistors 368 and 370, impedance 372 and VCA 374. The earphone impedance is modeled by the element Z in the schematic. At the output of the bridge the drive signal that is sent to the earphone is cancelled out to leave just the microphone signal component. For this to work effectively, the microphone signal must be amplified within the headset since it is normally small compared to the earphone signal, hence the need for op-amp 362.

This technique requires that earphone impedance be known or it will fail due to errors in the subtraction of the drive signal. Earphone resistance may be measured whenever the headset is plugged in to determine the earphone impedance. This may be accomplished by applying a small test signal to the headset, at a level sufficiently low so as to ensure the microphone electronics will not be activated, thus allowing a true measure of the earphone characteristics. The bridge circuit balance is then automatically adjusted by means of the voltage controlled amplifier 374 as shown in FIG. 22.

The resistance measurement may be extended to ensure compatibility with a normal headphone by making measurements with a high signal voltage. With a conventional earphone the current drawn will at all times be proportional to the voltage applied, but with the active headset the current will become non-linear as the microphone circuit kicks in, and this can be used for headset identification. The low frequency test signal is outside of the range of hearing so that it does not cause discomfort and is applied before a DC voltage is added. The DC voltage is only added after an active headset is detected. Alternatively, the technique previously discussed of using an ultrasonic signal and a predetermined impedance within the headset may be employed.

Yet another technique for combining the earphone and microphone connections is to make use of a radio frequency carrier signal as shown in FIG. 23. As described previously, a DC voltage may be added to the power amplifier output signal and used to power the microphone circuit. In this configuration, the microphone modulates either the amplitude, frequency, or phase of a high frequency oscillator 382, whose output is then capacitively coupled onto the earphone drive by capacitor 384, while being isolated from the microphone by RF inductor 386. The external device power amplifier is fed through an inductor to the output, thus blocking the RF signal from being shorted out by the power amplifier. The RF signal is then passed into demodulator 390 by capacitor 392 and demodulated to recover the microphone signal. A means of detecting a normal headset is used so that the DC level may be removed.

Many of the techniques previously described rely upon detecting the presence or absence of an active headset in order to appropriately configure the electronics inside the external device. This identification may be extended to identify the particular model of the active headset in addition to its presence. This would allow the cancellation controller transfer function to be adjusted to suit different active headset arrangements, for instance closed-back versus open-back, without having to measure the transfer function in situ and adapt the electronics accordingly. The headsets may be classified into types in much the same way as cassette tapes, with the type number referring to either performance level or acoustic configuration. This may be taken a step further to prevent the cancellation electronics within the external device from being activated unless a particular brand of headset is used.

The technique previously described for identifying the headset, whereby the headset is interrogated with an ultrasonic test tone (or multiple test tones) and a particular impedance looked for, or else a coded identification signal is generated within the headset itself may be used to identify the particular model of the active headset in addition to it's presence. This should be straightforward since, whether or not the majority of the electronics is incorporated within the headset itself, power is still needed for the microphone. Thus the headset can house an inexpensive ASIC, connected across the microphone, that injects a low level high frequency digital identification code onto the microphone signal which can be read by corresponding electronics within the external device. The code can either be present all the time or activated for just a short period each time the headset is powered up.

The present invention offers several advantages over the prior art. First, all of the noise cancellation circuitry for an active headset may be remote from the headset. Second, some of the noise cancellation circuitry for an active headset may be remote from the headset. Third, a stereo headset is provided with two stereo jack plugs or a six pin connector for connection to the noise cancellation circuitry without requiring replacement of the stereo jack socket by an eight pin socket. It should be noted that the present invention additionally provides for a seven pin connector in the case where it is desirable to use four connections to the headset, as in the prior art, but three connections to the microphone. Such a seven pin connector still uses one less pin than the prior art eight pin connector. Additionally, as previously discussed, three, four and five pin connectors are encompassed by the present invention as well and provide a significant advantage over the prior art eight pin connector. Finally, a transient detector may be provided in the noise cancellation circuitry to overcome noise in the ear due to the generation of transients when plugging in or unplugging the stereo jack plugs of an active headset.

While the invention has been particularly shown and described with reference to a preferred embodiment, it will be understood by those skilled in the art that various changes in form and detail may be made therein without departing from the spirit and scope of the invention. For instance, it will be understood by one of ordinary skill in the art that the terms headphone and earphone are used interchangeably.

What is claimed is:

1. A noise cancellation system having compatibility with existing socket configurations, comprising:
an active headset, having at least a first earphone, a first microphone, and a first gain control element that provides gain control of the first microphone;
a noise cancellation circuit that is located remotely from the active headset and comprises an amplifier circuit that is coupled to the first earphone of the active headset; and
a plurality of electrical connections having a maximum number of electrical connections for connecting the noise cancellation circuitry to the active headset that is less than the number of electric connections required in a conventional active headset.

2. A noise cancellation system according to claim 1, wherein the active headset is a stereo headset further comprising a second earphone, a second microphone, and a second gain control element that provide gain control of the of the second microphone, and wherein the maximum number of electrical connections is seven.

3. A noise cancellation system according to claim 1, wherein the active headset is a mono headset and wherein the maximum number of electrical connections is four.

4. A noise cancellation system according to claim 1, wherein the active headset is a mono headset comprising a boom microphone and wherein the maximum number of electrical connections is five.

5. A noise cancellation system according to claim 2, wherein the electrical connections comprises two stereo jack plugs.

6. A noise cancellation system according to claim 3, wherein the electrical connections comprises two mono jack plugs.

7. A noise cancellation system according to claim 3, wherein the electrical connections comprises a stereo jack plug.

8. A noise cancellation system according to claim 4, wherein the electrical connections comprise a stereo jack plug and a mono jack plug.

9. A noise cancellation system according to claim 2, wherein the electrical connections comprise a six pin connector.

10. A noise cancellation system according to claim 3, wherein the electrical connections comprise at least one of a three pin connector and a four pin connector.

11. A noise cancellation system according to claim 4, wherein the electrical connections comprise at least one of a four pin connector and a five pin connector.

12. A noise cancellation system according to claim 2, wherein the electrical connections comprises a seven pin connector.

13. A noise cancellation system according to claim 1, wherein the first microphone is coupled to a noise cancellation controller of the noise cancellation circuit at a first electrical connection of the plurality of electrical connections, the first microphone is coupled to a first voltage potential at a second electrical connection of the plurality of electrical connections, and said first microphone is coupled in parallel with said first gain control element.

14. A noise cancellation system according to claim 1, wherein a first current source of a noise cancellation controller of the noise cancellation circuit comprises:
a voltage source element; and
a bootstrapping capacitor coupled to the voltage source element and coupled to the first microphone to provide a correct bias voltage to said first microphone;
wherein the coupling of the bootstrapping capacitor to the voltage source element causes the voltage source element to be operable as a current source within an operational frequency range of the active headset.

15. A noise cancellation system according to claim 13, wherein the gain of the first microphone is adjustable by a first adjustment element that is the first gain control element.

16. A noise cancellation system according to claim 2, wherein the first microphone is coupled to the noise cancellation controller at a first electrical connection of the plurality of electrical connections, the first microphone is coupled to the second microphone and a first voltage potential at a second electrical connection of the plurality of electrical connections, the second microphone is coupled to the noise cancellation controller at a third electrical connection of the plurality of electrical connections, and said first and second microphones are coupled in parallel with said first gain control element.

17. A noise cancellation system according to claim 16, wherein the first voltage potential is ground potential.

18. A noise cancellation system according to claim 16, wherein the gain of the first microphone is adjustable by a first adjustment element that is the first gain control element and the gain of the second microphone is adjustable by a second adjustment element that is the second gain control element.

19. A noise cancellation system according to claim 2, wherein a circuit of the active headset comprises:
a resistive element coupled to the first earphone and the second earphone to define a first electrical connection of the plurality of electrical connections, wherein the first earphone is coupled to a second electrical connection of the plurality of electrical connections and the second earphone is coupled to a third electrical connection of the plurality of electrical connections;
a capacitive element, wherein the first microphone, the second microphone, and a first terminal of the capacitive element are coupled together to define a fourth electrical connection of the plurality of electrical connections;
a first adjustment element coupled to the first microphone at a fifth electrical connection of the plurality of electrical connections; and
a second adjustment element coupled to the second microphone at a sixth electrical connection of the plurality of electrical connections,
wherein the adjustment elements are coupled to the resistive element.

20. A noise cancellation system according to claim 1, wherein a circuit of the active headset comprises:
a resistive element coupled to the first earphone to define a first electrical connection of the plurality of electrical connections, wherein the first earphone is coupled to a second electrical connection of the plurality of electrical connections;
a capacitive element, wherein the first microphone, and a first terminal of the capacitive element are coupled together to define a third electrical connection of the plurality of electrical connections;
a first adjustment element coupled to the first microphone at a fourth electrical connection of the plurality of electrical connections; and
wherein the first potentiometer is coupled to the resistive element.

21. A noise cancellation system according to claim 2, wherein the first earphone has a first terminal and a second terminal and is characterized as having an impedance and the second earphone has a first terminal and a second terminal and is characterized as having an impedance; and
wherein a common contact in the amplifier circuit of the noise cancellation circuitry serves as an input connection to the first terminal of the first earphone and the first terminal of the second earphone.

22. A noise cancellation system according to claim 21, wherein the common contact is coupled to a voltage source.

23. A noise cancellation system according to claim 21, wherein the common contact is coupled to a voltage reference.

24. A noise cancellation system according to claim 21, the amplifier circuit further comprising:
a first amplifier connected to the first earphone that amplifies signals to the first earphone;
a second amplifier connected to the second earphone that amplifies signals to the second earphone; and
a third amplifier that couples the first earphone to the second earphone and is connected to the common contact, wherein the third amplifier acts as a virtual ground for the first earphone and the second earphone.

25. A noise cancellation system according to claim 1, the active headset further comprising:
a second microphone and a second gain control element that provides gain control for the second microphone.

26. A noise cancellation system according to claim 25, wherein the noise cancellation circuitry further comprises:
a first bootstrapped emitter follower transistor and a second bootstrapped emitter follower transistor which behave as current sources at audio frequencies and provide a correct bias voltage simultaneously to the first microphone and second microphone, respectively, wherein the first microphone of the first earpiece has a first terminal and a second terminal and the second microphone of the second earpiece has a first terminal and a second terminal, so that a common contact serves as an input connection to the first terminal of the first microphone and the first terminal of the second microphone.

27. A noise cancellation system according to claim 26, wherein the noise cancellation controller comprises:
a first current source that provides a first bias voltage to the first microphone; and
a second current source that provides a second bias voltage to the first microphone.

28. A noise cancellation system according to claim 27, wherein the first current source comprises:
a first emitter-follower transistor having a base provided with the first bias voltage, a collector coupled to a second voltage potential, and an emitter coupled to a first electrical connection of the plurality of electrical connections; and
a first bootstrapping capacitor that couples the base of the first emitter-follower transistor to the first electrical connection of the plurality of electrical connections; and wherein the second current source comprises:
a second emitter-follow transistor having a base provided with the second bias voltage, a collector coupled to the second voltage potential, and an emitter coupled to the third electrical connection of the plurality of electrical connections; and
a second bootstrapping capacitor that couples the base of the second emitter-follower transistor to the third electrical connection of the plurality of electrical connections.

29. A noise cancellation system capable of preventing noise transients that result upon plugging and unplugging a headset according to claim 28, wherein the noise cancellation circuit has a transient detector capable of preventing noise transients that result upon plugging and unplugging the active headset, wherein the transient detector comprises:
a window comparator; and
a mute logic circuit that mutes an output signal of the noise cancellation circuit received by the first earphone and the second earphone when the output signal exceeds a predetermined amplitude level.

30. A noise cancellation system according to claim 1, wherein the noise cancellation controller comprises:
a first current source that provides a first bias voltage to the first microphone.

31. A noise cancellation system according to claim 30, wherein the first current source comprises:
a first emitter-follower transistor having a base provided with the first bias voltage, a collector coupled to a second voltage potential, and an emitter coupled to the first electrical connection of the plurality of electrical connections; and
a first bootstrapping capacitor that couples the base of the first emitter-follower transistor to the first electrical connection of the plurality of electrical connections.

32. A noise cancellation system capable of preventing noise transients that result upon plugging and unplugging a headset according to claim 31, wherein the noise cancellation circuit has a transient detector capable of preventing noise transients that result upon plugging and unplugging the active headset, wherein the transient detector comprises:
a window comparator; and
a mute logic circuit that mutes an output signal of the noise cancellation circuit received by the first earphone when the output signal exceeds a predetermined amplitude level.

33. A noise cancellation system according to claim 25, wherein the noise cancellation circuitry further comprises:
a first operational amplifier and a second operational amplifier which act as current sources over the operating frequency range of the active headset and also provide correct bias voltage simultaneously to the first microphone and second microphone, respectively, wherein the first microphone of the first earpiece has a first terminal and a second terminal and the second microphone of the second earpiece has a first terminal and a second terminal, so that a common contact serves as an input connection to the first terminal of the first microphone and the first terminal of the second microphone.

34. A noise cancellation system according to claim 1, wherein the noise cancellation circuitry further comprises:
a first operational amplifier which acts as a current sources over the operating frequency range of the active headset and also provides a correct bias voltage to the first microphone, wherein the first microphone of the first earpiece has a first terminal and a second terminal and the second microphone of the second earpiece has a first terminal and a second terminal.

35. A noise cancellation system according to claim 1, wherein the noise cancellation circuit is integral to a passenger cabin of a vehicle.

36. A noise cancellation system according to claim 35, wherein the noise cancellation circuit is provided in an arm rest of an aircraft seat.

37. A noise cancellation system according to claim 36, wherein the arm rest is provided with a first stereo jack plug socket and a second stereo jack plug socket.

38. A noise cancellation system according to claim 37, wherein the active headset has a first earpiece and a second earpiece, with the first earpiece having a first earphone and a first microphone and the second earpiece having a second earphone and a second microphone, wherein the first stereo jack plug socket provides for three input electrical connections of the plurality of electrical connections to the first microphone in the first earpiece and the second microphone in the second earpiece and the second stereo jack plug socket provides three input electrical connections to the first earphone in the first earpiece and the second earphone in the second earpiece.

39. A noise cancellation system according to claim 1, wherein the remote noise cancellation circuit is provided in an arm rest of an aircraft seat and the arm rest is provided with a six contact socket.

40. A noise cancellation system according to claim 1, wherein the noise cancellation circuit includes a transient detector.

41. A noise cancellation system according to claim 1,
wherein the transient detector includes a window comparator and a mute logic circuit for muting at least a first signal to at least the first earphone when the at least first signal exceeds a predetermined amplitude.

42. A noise cancellation system according to claim 1, said active headset further comprising:
a microphone terminal of the first microphone;
a headphone terminal of the first headphone at a direct current potential with respect to the microphone terminal; and
a decoupling network operable on a signal at the headphone terminal to supply power for the first microphone and the first gain control element; wherein a Wire-ORed output of the first microphone and the first gain control element is AC coupled to a microphone output terminal of the active headset.

43. A noise cancellation system, comprising:
an active headset, having at least a first earphone, a first microphone, and a first gain control element for controlling the gain of the first microphone;
a noise cancellation circuit which is contained within the active headset; and
a connection means having four contacts for connecting the noise cancellation circuit to the active headset, wherein the four contacts are used to power the noise cancellation circuit contained within the active headset.

44. A noise cancellation system according to claim 43, wherein the connection means is a stereo plug and socket pair, with the stereo plug having an extended length and a contact at the end of the extended length stereo plug that is beyond the distance that a stereo plug would normally extend and with the socket having a contact operatively configured for receiving power from the contact on the stereo plug and providing power to the active circuitry.

45. A noise cancellation system according to claim 44, wherein the stereo plug is compatible with existing socket configurations.

46. A noise cancellation system according to claim 45, wherein the stereo plug has a plurality of contacts and a length that is switchable between a conventional stereo plug length and an extended stereo plug length and wherein the contacts of the stereo plug are reconfigured to provide power when the stereo plug is the extended stereo plug length.

47. A noise cancellation system having compatibility with existing socket configurations, comprising:
an active headset, having at least a first earphone, a first microphone, and a first gain control element for controlling a gain of the first microphone;
a noise cancellation circuit, at least a portion of which is contained within the active headset;
a plurality of electrical connections for connecting the active headset to the noise cancellation circuit wherein the electrical connections are compatible with standard audio equipment; and
a powering means, remote from the active headset, that provides power to the portion of the noise cancellation circuit that is contained within the active headset.

48. A noise cancellation system according to claim 47, wherein the powering means comprises:
an external device having a battery and a dual-function power supply socket;
an active headset power plug; and
wherein the dual-function power supply socket is operatively configured to receive power for the external device when a power source is plugged into the dual-function power supply socket and to provide power to the noise cancellation circuit from the battery when the active headset power plug is plugged into the dual-function power supply socket.

49. A noise cancellation system according to claim 48, wherein the battery does not supply power to the external device when a power source is plugged into the power supply socket.

50. A noise cancellation system according to claim 47, wherein the powering means comprises:
an external device having a battery and a dual-function power supply socket;
an active headset power plug; and
wherein the active headset power plug has a rear power socket and is configured to provide power to the external device and the noise cancellation circuit from a power source when the power source is plugged into the rear power socket of the active headset power plug.

51. A noise cancellation system according to claim 47, wherein the powering means comprises:
an external device having a battery and a line out socket;
an active headset power plug; and
wherein the line out socket is operatively configured to automatically switch to provide power to the noise cancellation circuitry from the battery when the active headset power plug is plugged into the line out socket.

52. A noise cancellation system according to claim 51, wherein the powering means further comprises a sensing system that uses an ultrasonic test tone to determine when the line out socket is switched to provide power to the noise cancellation circuitry.

53. A noise cancellation system according to claim 51, wherein the powering means further comprises a sensing system that monitors current drawn by the active headset to determine when the line out socket is switched to provide power to the noise cancellation circuitry.

54. A noise cancellation system according to claim 51, wherein the powering means further comprises a sensing system within the active headset that has an application specific integrated circuit which injects a digital identification code onto a microphone signal that can be read by an external device in order to determine when the line out socket is switched to provide power to the noise cancellation circuitry.

55. A noise cancellation system according to claim 47, wherein the powering means comprises an external device having a battery and a plurality of retractable contacts on the connection means and wherein the retractable contacts are operatively configured to provide power to the noise cancellation circuitry from the battery when an active headset is connected to the external device.

56. A noise cancellation system according to claim 47, wherein the powering means uses phantom powering to provide power to the noise cancellation circuitry wherein the presence of the active headset is sensed so that direct current is not applied to a passive headset.

57. A noise cancellation system according to claim 56, wherein the phantom powering is disabled when a non-active headset is used.

58. A noise cancellation system according to claim 57, wherein the powering means further comprises a sensing system that uses an ultrasonic test tone to determine when the non-active headset is used and the phantom powering is disabled.

59. A noise cancellation system according to claim 57, wherein the powering means further comprises a sensing system that monitors current drawn by the active headset to determine when the non-active headset is used and the phantom powering is disabled.

60. A noise cancellation system according to claim 57, wherein the powering means further comprises a sensing system within the active headset that has an application specific integrated circuit which injects a digital identification code onto a microphone signal that can be read by an external device in order to determine when the non-active headset is used and the phantom powering is disabled.

61. A noise cancellation system according to claim 47, wherein the powering means is a superimposed positive DC voltage on a conventional analog output to provide power to the noise cancellation circuitry.

62. A noise cancellation system according to claim 47, wherein the powering means comprises a pulse width modulator circuit that is modulated by an audio signal and that produces a square wave having a higher frequency than the desired audio frequency and a rectification and storage means,
wherein when the active headset is plugged into the noise cancellation circuit, the square wave produced by the pulse width modulator circuit is rectified and filtered by the rectification and storage means to produce a power signal and is filtered to recover the audio signal.

63. The noise cancellation system of claim 47, wherein the pulse width modulator circuit is disabled upon detection of a passive headset being plugged into the noise cancellation circuit.

64. A noise cancellation system according to claim 63, wherein the powering means further comprises a sensing system that uses an ultrasonic test tone to detect when the passive headset is plugged into the noise cancellation circuit.

65. A noise cancellation system according to claim 63, wherein the powering means further comprises a sensing system that monitors current drawn by the active headset to detect when the passive headset is plugged into the noise cancellation circuit.

66. A noise cancellation system according to claim 63, wherein the powering means further comprises a sensing system within the active headset that has an application specific integrated circuit which injects a digital identification code onto a microphone signal that can be read by an external device in order to detect when the passive headset is plugged into the noise cancellation circuit.

67. A noise cancellation system according to claim 47, wherein the powering means comprises an audio signal produced by an external device, wherein the volume of the audio signal is maximized and then rectified to charge an energy storage element so that the active headset is powered by the audio signal during its peaks and by the energy storage element otherwise.

68. A noise cancellation system according to claim 67, wherein the energy storage element is a reservoir capacitor.

69. A noise cancellation system according to claim 67, wherein the energy storage element is a battery.

70. A noise cancellation system according to claim 67, wherein the audio signal is rectified by a diode element and the energy storage element is charged through a current limiting resistor.

71. A noise cancellation system according to claim 67, wherein a switched-mode power supply is used to boost the audio signal level so that the energy storage element may be charged even when the voltage of the audio signal is less than the voltage of the energy storage element.

72. A noise cancellation system according to claim 67, wherein distortion introduced by the resistance of a cable of the active headset may be reduced by a linearized charging circuit that provides a constant load impedance in order to reduce distortion of the audio signal.

73. A noise cancellation system having compatibility with existing socket configurations, comprising:
an active headset, having at least a first earphone, a first microphone, and a gain control element for controlling a gain of the first microphone;
a noise cancellation circuit, a portion of which is contained within an external device;
a plurality of electrical connections for connecting the active headset to the noise cancellation circuit; and
a powering means that provides power to the portion of the noise cancellation circuit that is contained within the external device.

74. A noise cancellation system according to claim 73, wherein the external device is a consumer stereo equipment.

75. A noise cancellation system according to claim 73, wherein the powering means comprises a dual-function socket of the external device, wherein when the active headset is plugged into the dual-function socket of the external device, the dual-function socket serves an auxiliary function.

76. A noise cancellation system according to claim 75, wherein the powering means further comprises a sensing system that uses an ultrasonic test tone to determine when the dual-function socket is switched to the auxiliary function.

77. A noise cancellation system according to claim 75, wherein the powering means further comprises a sensing system that monitors current drawn by the active headset to determine when the dual-function socket is switched to the auxiliary function.

78. A noise cancellation system according to claim 75, wherein the powering means further comprises a sensing system within the active headset that has an application specific integrated circuit which injects a digital identification code onto a microphone signal that can be read by an external device in order to determine when the dualfunction socket is switched to the auxiliary function.

79. A noise cancellation system according to claim 73, wherein the powering means comprises a pulse width modulator circuit that is modulated by an audio signal and that produces a square wave having a higher frequency than the desired audio frequency and a rectification and storage means,
wherein when the active headset is plugged into the noise cancellation circuit, the square wave produced by the pulse width modulator circuit is rectified and filtered by the rectification and storage means to produce a power signal and is filtered to recover the audio signal, and
wherein during a positive portion of the square wave, the first earphone and the second earphone of the active headset are disconnected and an output signal of a microphone of the active headset is simultaneously connected into the external device so that the output signal of the microphone can be measured.

80. The noise cancellation system of claim 79, wherein the pulse width modulator circuit is disabled upon detection of a passive headset being plugged into the noise cancellation circuit.

81. A noise cancellation system according to claim 80, wherein the powering means further comprises a sensing system that uses an ultrasonic test tone to detect when the passive headset is plugged into the noise cancellation circuit.

82. A noise cancellation system according to claim 80, wherein the powering means further comprises a sensing system that monitors current drawn by the active headset to detect when the passive headset is plugged into the noise cancellation circuit.

83. A noise cancellation system according to claim 80, wherein the powering means as further comprises a sensing system within the active headset that has an application specific integrated circuit which injects a digital identification code onto a microphone signal that can be read by an external device in order to detect when the passive headset is plugged into the noise cancellation circuit.

84. The noise cancellation system according to claim 73, wherein the powering means comprises superimposing an audio signal upon a positive DC voltage level to produce a resultant signal having a voltage level that is maintained at a positive voltage potential,
wherein the audio signal is filtered out of the resultant signal to produce a DC signal that powers the portion of the noise cancellation circuit, and
wherein a plurality of high frequency negative-going spikes are superimposed upon a drive signal and during the plurality of negative-going spikes the first earphone and the second earphone of the active headset are disconnected and an output signal of a microphone of the active headset is simultaneously connected into the external device so that the output signal of the microphone can be measured.

85. A noise cancellation system according to claim 84, wherein the audio signal is filtered out by a resistive element and a capacitive element.

86. A noise cancellation system according to claim 73, wherein the powering means adds a first DC offset to a first drive signal of the first earphone and the first earphone is capacitor coupled to remove the first DC offset, and
wherein the powering means includes a bridge circuit in the external device that operates to separate a first microphone signal from the first drive signal, and
wherein the impedance of the first earphone is measured when the active headset is plugged-in in order to enable the bridge circuit to cancel the first drive signal, leaving the first microphone signal.

87. A noise cancellation system according to claim 86, wherein a test signal is used to measure the impedance of the first earphone and the impedance of the second earphone when the active headset is plugged-in.

88. A noise cancellation system according to claim 73, wherein the active headset is a stereo headset further comprising a second earphone and wherein the powering means comprises:
a DC voltage upon which a radio frequency carrier employing a high frequency oscillator is imposed in the active headset, wherein the radio frequency carrier is modulated by a microphone output signal of a microphone and demodulated in the external device to recover the microphone output signal, the output signal of the high frequency oscillator is capacitively coupled to a drive signal of the first earphone and a drive signal of the second earphone, and the radio frequency carrier is blocked from a power source of the microphone by a radio frequency inductive element.

89. A noise cancellation system having compatibility with existing socket configurations, comprising:
an active headset, having an earphone and a microphone coupled to a common ground;
a noise cancellation circuit, a portion of which is contained within an external device;
a plurality of electrical connections for connecting the active headset to the noise cancellation circuit; and
a powering means that provides power to the portion of the noise cancellation circuit that is contained within the external device,
wherein crosstalk between a earphone signal produced by the earphone and a microphone signal produced by the microphone is eliminated by placing a resistive element between the common ground and circuit ground, sensing a voltage drop across the resistive element, and subtracting a proportion of the voltage drop from the microphone signal necessary to eliminate the crosstalk.

90. A transient elimination circuit contained within a headset, comprising:
a first resistive element having a first terminal and a second terminal, with the first terminal coupled to a headphone common;
a potentiometer having a first terminal and a second terminal;
a decoupling capacitor having a first terminal and a second terminal, with the first terminal of the decoupling capacitor coupled to a second terminal of the first resistive element and a first terminal of the potentiometer;
a coupling capacitor having a first terminal and a second terminal;
a microphone having a first terminal and a second terminal, with the first terminal of the microphone coupled to the second terminal of the potentiometer and the first terminal of the coupling capacitor; and
a second resistive element having a first terminal and a second terminal, with the first terminal of the second resistive element coupled to the second terminal of coupling capacitor to form a microphone output signal and with the second terminal of the second resistive element coupled to the second terminal of the decoupling capacitor and the second terminal of the microphone to form a microphone common,
wherein the headphone common is at a DC voltage potential with respect to the microphone common and is filtered to provide power to the microphone, and
wherein when the headset is plugged or alternately unplugged, the decoupling capacitor is slowly charged or alternately discharged through the first resistive element so as to not cause a transient condition.
